(19) United States
(12) Patent Application Publication
(10) Pub. No.: US 2021/0313053 A1
(43) Pub. Date: Oct. 7, 2021

(54) MEDICAL INFORMATION PROCESSING SYSTEM, MEDICAL INFORMATION PROCESSING DEVICE, AND MEDICAL INFORMATION PROCESSING METHOD

(71) Applicant: SONY CORPORATION, Tokyo (JP)

(72) Inventors: Takayoshi HIRAI, Tokyo (JP); Yukako FUJIMOTO, Tokyo (JP); Kento NAKADA, Tokyo (JP)

(73) Assignee: SONY CORPORATION, Tokyo (JP)

(21) Appl. No.: 17/266,400

(22) PCT Filed: Aug. 14, 2019
(86) PCT No.: PCT/JP2019/031987

§ 371 Date: Feb. 5, 2021

(30) Foreign Application Priority Data
Aug. 15, 2018 (JP) 2018-152969

(51) Int. Cl. G16H 40/20; G16H 50/30

(52) U.S. Cl. CPC G16H 40/20; G16H 50/30

(57) ABSTRACT

There is provided a medical information processing system that includes: an acquisition unit (110) that acquires an examination arrangement request that includes examination type information that indicates a type of medical-related examination, and user information regarding a user; a presentation unit (123) that presents, to the user, an examination resource capable of performing the examination, based on the examination arrangement request and the user information; a management unit (222) that manages a result of the examination performed on the user in association with the user information; and an output unit (224) that outputs the result based on input performed by the user.

### TECHNICAL FIELD

The present disclosure relates to a medical information processing system, a medical information processing device, and a medical information processing method.

### BACKGROUND ART

In recent years, increases in medical expenses have become a problem, and there is demand for a method for suppressing an increase in medical expenses. For example, PTL 1 listed below proposes a technique for making it possible to detect whether or not a person who has been instructed to undergo a medical consultation at a medical institution is actually undergoing a medical examination or treatment at the medical institution, and re-instruct the patient if necessary in order to prevent the worsening of symptoms, thus suppressing an increase in medical expenses.

### CITATION LIST

### Patent Literature

### [PTL 1]

### JP 2004-164173A

### SUMMARY

### Technical Problem

However, with the technique described in PTL 1 for example, a medical consultation at a medical institution is often required, and therefore the technique described in PTL 1 for example is insufficient as a solution for an increase in medical expenses. For example, when a user desires to undergo a medical examination, the user is often required to undergo a medical consultation that includes an examination at a medical institution such as a hospital. Therefore, medical institutions are required to possess a considerable amount of resources (e.g., doctors and equipment), which leads to an increase in medical expenses.

Therefore, the present disclosure has been made in view of the foregoing, and the present disclosure provides a novel and improved medical information processing system, medical information processing device, and medical information processing method capable of more appropriately suppressing an increase in medical expenses.

### Solution to Problem

According to the present disclosure, it is possible to provide a medical information processing system that includes: an acquisition unit configured to acquire an examination arrangement request that includes examination type information that indicates a type of medical-related examination, and user information that is information regarding a user; a presentation unit configured to present, to the user, an examination resource capable of performing the examination, based on the examination arrangement request and the user information; a management unit configured to manage a result of the examination performed on the user in association with the user information; and an output unit configured to output the result based on input performed by the user.

Also, according to the present disclosure, it is possible to provide a medical information processing device that includes: an acquisition unit configured to acquire an examination arrangement request that includes examination type information that indicates a type of medical-related examination, and user information that is information regarding a user; a presentation unit configured to present, to the user, an examination resource capable of performing the examination, based on the examination arrangement request and the user information; a management unit configured to manage a result of the examination performed on the user in association with the user information; and an output unit configured to output the result based on input performed by the user.

Also, according to the present disclosure, it is possible to provide a medical information processing method executed by a computer, the method comprising: acquiring an examination arrangement request that includes examination type information that indicates a type of medical-related examination, and user information that is information regarding a user; presenting, to the user, an examination resource capable of performing the examination, based on the examination arrangement request and the user information; managing a result of the examination performed on the user in association with the user information; and outputting the result based on input performed by the user.

The present disclosure makes it possible to more appropriately suppress an increase in medical expenses by performing a medical examination more appropriately and using results of the examination more appropriately.

### Advantageous Effects of Invention

As described above, according to the present disclosure, it is possible to more appropriately suppress an increase in medical expenses.

Note that the above effects are not necessarily limiting, and any of the effects shown in the present specification as well as other effects that can be ascertained from the present specification may be realized together with the above effects or in place of the above effects.

### BRIEF DESCRIPTION OF DRAWINGS

FIG. 1 is a block diagram showing an example of a system configuration of a medical information processing system according to a first embodiment.

FIG. 2 is a block diagram showing an example of a functional configuration of a matching server 100.

FIG. 3 is a block diagram showing an example of a functional configuration of a management server 200.

FIG. 4 is a block diagram showing an example of a functional configuration of a user terminal 400.

FIG. 5 is a sequence diagram showing an example of a flow of processing up to the arrangement of examination resources.

FIG. 6 is a sequence diagram showing an example of a flow of processing up to the arrangement of examination resources.

FIG. 7 is a sequence diagram showing an example of the flow of examination result output processing.

FIG. 8 is a block diagram showing an example of a system configuration of a medical information processing system according to a second embodiment.

FIG. 9 is a block diagram showing an example of a functional configuration of a diagnosis server 600.

FIG. 10 is a sequence diagram showing an example of the flow of processing related to the provision of comprehensive result information to a user.

FIG. 11 is a diagram showing a specific example of a medical inquiry AI user interface.

FIG. 12 is a diagram showing a specific example of a medical inquiry AI user interface.

FIG. 13 is a diagram showing a specific example of a medical inquiry AI user interface.

FIG. 14 is a diagram showing a specific example of a medical inquiry AI user interface.

FIG. 15 is a diagram showing a specific example of a medical inquiry AI user interface.

FIG. 16 is a diagram showing a specific example of a medical inquiry AI user interface.

FIG. 17 is a diagram showing a specific example of a medical inquiry AI user interface.

FIG. 18 is a block diagram showing an example of a hardware configuration of an information processing device 900 that realizes a matching server 100, a management server 200, an examination terminal 300, a user terminal 400, and a diagnosis server 600.

### DESCRIPTION OF EMBODIMENTS

Preferred embodiments of the present disclosure will be described in detail below with reference to the accompanying drawings. Note that in the present specification and drawings, constituent elements having substantially the same functional configuration are designated by the same reference numerals and thus will not be described redundantly.

Descriptions will be given in the following order.

### 1. First Embodiment

### 1.1. System Configuration

### 1.2. Functional Configuration of Device

1.3. Processing flow

### 2. Second Embodiment

### 2.1. System Configuration

### 2.2. Functional Configuration of Device

2.3. Processing flow

### 3. User Interface

### 4. Hardware Configuration

### 5. Summary

### 1. First Embodiment

### (1.1. System Configuration)

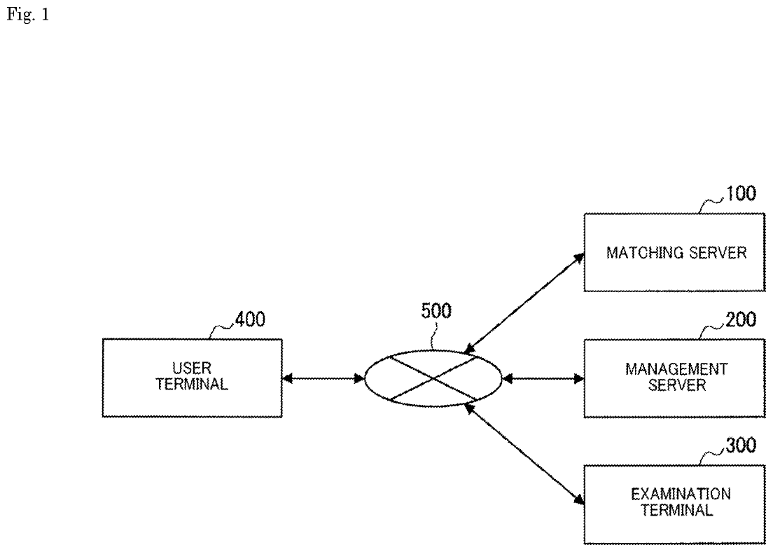

First, an example of the system configuration of a medical information processing system according to a first embodiment of the present disclosure will be described with reference to FIG. 1.

As shown in FIG. 1, the medical information processing system according to the present embodiment includes a matching server 100, a management server 200, an examination terminal 300, and a user terminal 400, and these devices are connected by a network 500.

### (Matching Server 100)

The matching server 100 is a medical information processing device for matching a user who is to undergo an examination with an examination resource for performing the examination. More specifically, the matching server 100 first acquires an examination arrangement request, which includes examination identification information that indicates a certain type of medical examination (hereinafter, sometimes called an “examination” for convenience), and user information, which is information regarding a user, from the user terminal 400.

Here, “medical examination” overall refers to actions performed to evaluate the state of the user or injury/illness, and refers to actions performed separately from diagnosis, treatment, surgery, and estimation of user symptoms. More specifically, “medical examination” includes an examination for evaluating the physical condition of the user, an examination for evaluating the presence/absence or extent of a specific injury/illness, an examination performed by the user or performed by a medical examination worker with use of a predetermined examination device. Then, after the medical examination is performed, diagnosis, treatment, surgery, estimation of user symptoms, or the like is performed by a doctor based on the result of the medical examination (hereinafter, sometimes called the “examination result”). Note that if the examination is an X-ray test, the examination result is X-ray image information or the like, and if the examination is a blood test, the examination result is any of various numerical values regarding blood levels. “Medical examination” may also include an examination carried out under the supervision of a doctor, a dentist, a nurse, or a clinical laboratory technician. Also, data indicating an examination result may be associated with information regarding the device or apparatus used in the examination, information regarding the examiner who performed the examination, information regarding the examination institution, or the like. Moreover, metadata that indicates the type of medical data may be attached to the data that indicates the examination result.

Also, “type of examination (hereinafter, sometimes called the ‘examination type’)” indicated by examination type information refers to something that identifies an examination, and may be replaced with any term that indicates something that identifies an examination, such as an examination type, division, classification, or category.

Also, “user information” refers to any information regarding the user who is to undergo an examination. In the present embodiment, the user information is envisioned to include user identification information (e.g., an ID), user attribute information (e.g., name, gender, age, blood type, address, telephone number, family structure, occupation, insurance information, and credit card information), user location information (e.g., latitude, longitude, altitude), historical information about examinations that the user underwent in the past (e.g., the date/time or duration for which the user underwent an examination and the examination content), setting information related to an examination that the user is to undergo in the future (e.g., the user's requests, needs, or restrictions related to an examination), and the like, but the user information is not necessarily limited to such information. Note that if the user terminal 400 is a device carried by the user such as a smartphone or a device worn by the user such as a wearable device, the user position information is equivalent to the position of the user terminal 400 and therefore may be treated as “terminal information of the user terminal 400”.

Based on the examination arrangement request and the user information that were acquired, the matching server 100 then presents, to the user, examination resources available for performing examinations.

Here, “examination resource” in the present embodiment is envisioned to include a medical examination institution, a medical examination worker, an examination device (including an examination device such as a wheeled vehicle or a flying vehicle that has a movement function and can move to the user's position), and a test kit for use by the user, but the examination resources are not necessarily limited to these examples. Also, as mentioned above, “medical examination” refers to something that is done separately from diagnosis, treatment, surgery, and estimation of user symptoms, and therefore an examination resource is envisioned to be something not for a diagnosis, treatment, surgery, or estimation of user symptoms based on an examination result, but this term is not necessarily limited in this way. For example, at a hospital, which is an examination resource, a diagnosis or the like may be made as necessary based on an examination result obtained after a user examination. Through the above-described processing, the user can easily undergo an appropriate examination.

### (Management Server 200)

The management server 200 is a medical information processing device that manages examination results and examination resources. More specifically regarding the management of examination results, the management server 200 receives an examination result from the examination terminal 300 after an examination has been performed using an examination resource, and manages the examination result in association with user information received from the matching server 100. The management server 200 also outputs the examination result to a user-desired device based on input from the user.

As previously described, conventionally, when a user desires to undergo a medical examination, it is often necessary to undergo a medical consultation at a medical institution such as a hospital, thus leading to an increase in medical expenses. However, with the medical information processing system according to the present embodiment, the user can cause an examination result to be output to a desired device by inputting an instruction to the management server 200, and thus can take various actions based on the examination result. For example, the user can make a self-diagnosis by outputting the examination result to their own device, or can use a diagnostic service by outputting the examination result to a device of a diagnostic service provider. This therefore reduces the number of hospital visits and the like by users who have mild symptoms, and reduces diagnostic personnel expenses for example, thus making it possible to suppress an increase in medical expenses.

More specifically regarding the management of examination resources by the management server 200, the management server 200 performs communication with an examination resource, a device that manages the examination resource (e.g., a server of a medical examination institution), or the like in order to receive information regarding operation plans for the examination resource (or information regarding the operational status of an examination resource, for example), and manages such information. If there are two or more examination resources, the management server 200 manages such information for each of the examination resources.

The management server 200 can also makes arrangements for an examination resource. For example, the management server 200 can reserve an examination resource in an available time slot. Not only is the examination resource presented to the user, but also an arrangement for the examination resource is made, and therefore the user can undergo an examination more easily.

### (Examination Terminal 300)

The examination terminal 300 is a medical information processing device that transmits an examination result to the management server 200. More specifically, the examination terminal 300 is a device that is operated by a medical examination worker, and transmits an examination result to the management server 200 when the medical examination worker performs an operation (or automatically) after an examination has ended. Note that if the user performs the examination on their own with use of a test kit, the examination terminal 300 is a device operated by the user. Also, the examination terminal 300 may be the examination device (or test kit) used to perform the examination. Moreover, if the examination result is provided to the management server 200 by communication with the user terminal 400 or by another method (e.g., through the mail), the medical information processing system is not required to include the examination terminal 300.

### (User Terminal 400)

The user terminal 400 is an information processing device that transmits an examination arrangement request and user information. For example, the user terminal 400 provides a predetermined user interface to the user by executing a predetermined program. When the user performs various input via the user interface, the user terminal 400 generates an examination arrangement request that includes examination type information based on the input, and generates user information.

More specifically regarding the generation of the examination arrangement request, the user may be able to select a desired examination type from among multiple options, or may be able to directly input an examination type in a text format. Also, a configuration is possible in which the user inputs still image information or video information that shows an affected part, and the user terminal 400 analyzes the information and outputs an appropriate examination type or candidates for the same. A configuration is also possible in which the user answers multiple questions, and the user terminal 400 outputs an appropriate examination type or candidates for the same based on the responses. Given that it is not easy for the user to input an appropriate examination type, according to the above methods, the user terminal 400 can generate an appropriate examination arrangement request without placing a large burden on the user.

### (Network 500)

The network 500 is a network that connects the above-described devices through predetermined communication. Note that the network 500 does not necessarily need to connect all of the devices to each other, and may connect only devices that can communicate with each other. For example, it is possible for the examination terminal 300 to not be able to communicate with the user terminal 400 or the matching server 100, for example.

There are no particular limitations on the communication system, type of communication line, and the like used in the network 500. For example, the network 500 may be realized by a dedicated network such as an IP-VPN (Internet Protocol-Virtual Private Network). Also, the network 500 may be realized by a public network such as the Internet, a telephone line network, a satellite communication network, any of various LANs (Local Area Networks) including Ethernet (registered trademark), a WAN (Wide Area Network), or the like. Further, the network 500 may be realized by a wireless communication network such as a Wi-Fi (registered trademark) or Bluetooth (registered trademark) network.

An example of the system configuration of the medical information processing system according to the present embodiment has been described above. Note that the above system configuration described with reference to FIG. 1 is merely an example, and the system configuration of the medical information processing system according to the present embodiment is not limited to such an example. For example, some or all of the functions of the matching server 100 may be provided in the management server 200. Conversely, some or all of the functions of the management server 200 may be provided in the matching server 100. The system configuration of the medical information processing system of the present embodiment can be flexibly modified according to specifications and operations.

### (1.2. Functional Configuration of Device)

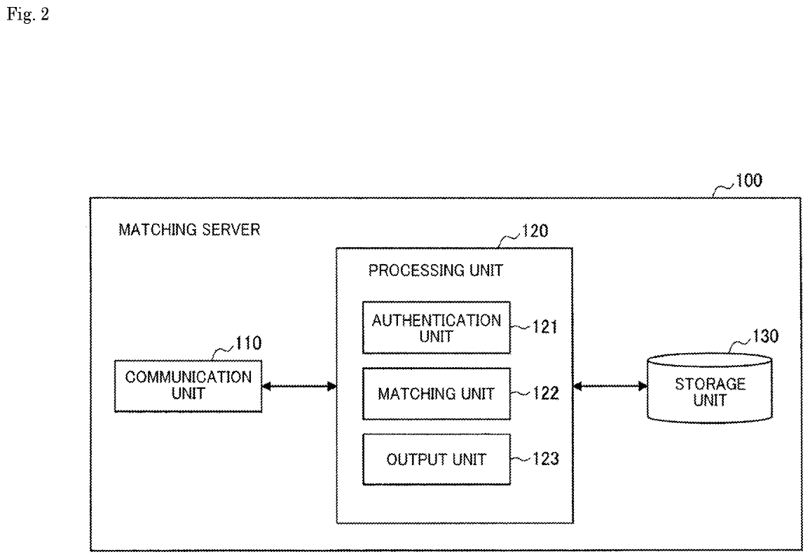

Next, an example of the functional configurations of devices included in the medical information processing system will be described with reference to FIGS. 2 to 4.

### (Example of Functional Configuration of Matching Server 100)

First, an example of the functional configuration of the matching server 100 will be described with reference to FIG. 2. As shown in FIG. 2, the matching server 100 includes a communication unit 110, a processing unit 120, and a storage unit 130. Further, the processing unit 120 includes an authentication unit 121, a matching unit 122, and an output unit 123.

The communication unit 110 has a functional configuration for communicating with an external device. For example, the communication unit 110 receives an examination arrangement request, user information, input information used for user authentication, and the like from the user terminal 400 (i.e., the communication unit 110 functions as an acquisition unit that acquires an examination arrangement request and user information). The communication unit 110 also transmits a result of examination resource matching processing, a result of examination resource arrangement processing, a result of user authentication, and the like to the user terminal 400. The communication unit 110 also transmits, to the management server 200, information regarding an examination resource for which an arrangement is to be made. The communication unit 110 also receives an examination resource arrangement result or the like from the management server 200. Note that the information transmitted by the communication unit 110 and cases where communication is performed are not limited to the above examples.

The processing unit 120 is a functional configuration for performing overall control of all processing performed by the matching server 100. For example, the processing unit 120 can control the start and stop of functional configurations. Note that there are no particular limitations on the processing content performed by the processing unit 120. For example, the processing unit 120 may control processing generally performed by various servers, general-purpose computers, PCs (Personal Computers), tablet PCs, and the like (e.g., processing related to an OS (Operating System)).

The authentication unit 121 is a functional configuration for performing user authentication. More specifically, if input information for use in user authentication is provided by the user terminal 400, the authentication unit 121 performs predetermined user authentication processing with use of the input information. Note that there are no particular limitations on the content of the user authentication processing. For example, the authentication unit 121 performs authentication with use of user identification (e.g., an ID) and a password, or biometric authentication with use of user biometric information. Accordingly, the authentication unit 121 can prevent access from an unauthorized third party. Biometric information is fingerprint information, finger vein information, facial information, or the like.

The matching unit 122 is a functional configuration for performing matching between a user and an examination resource based on an examination arrangement request and user information. More specifically, the matching unit 122 extracts, from the storage unit 130, examination resources capable of performing an examination that corresponds to the examination type in examination type information included in the examination arrangement request. For example, the storage unit 130 has a table in which examination resources are associated with examination type information, and the matching unit 122 extracts, from the table, the examination resources that are associated with information that matches the examination type information included in the examination arrangement request. Also, if the examination arrangement request includes information other than the examination type information, the matching unit 122 may perform matching processing based on such information. For example, if the examination arrangement request includes an examination priority level (or urgency level, for example), and the priority level indicates high value, the matching unit 122 may prioritize performing matching processing on that examination arrangement request over another examination arrangement requests (e.g., may interrupt other processing to perform matching processing, or change to (exchange) an examination resource that has already been matched for another examination arrangement request).

Furthermore, the matching unit 122 extracts a more appropriate examination resource based on the user information from among the examination resources that were extracted based on the examination arrangement request. For example, if the user's position information (e.g., latitude, longitude, and altitude) is included in the user information, the matching unit 122 may extract an examination resource that is located within a predetermined distance from the user's position, may extract a predetermined number of examination resources in the order of proximity to the user's position, or may extract the examination resource that is closest to the user's position. Note that the positions of examination resources are stored in advance in the storage unit 130 as examination resource position information. Also, if the user information includes historical information regarding examinations that the user has undergone in the past (e.g., the date/time or duration for which the user underwent an examination and the examination content), the matching unit 122 may extract an examination resource by which the user underwent an examination in the past, or may extract an examination resource by which the user has frequently undergone an examination. Also, if the user information includes setting information regarding an examination that the user is to undergo in the future (e.g., the user's requests, needs, or restrictions related to an examination, which may be included in the examination arrangement request), the matching unit 122 may use the setting information as extraction conditions. For example, if a region, a date/time, or the like have been set for an examination that the user wishes to undergo, the matching unit 122 may use the region, a date/time, or the like as an extraction condition when extracting an examination resource. Through such extraction processing, the matching unit 122 can extract an examination resource that can be easily utilized by the user. Note that although it has been described above that the matching unit 122 extracts examination resources based on the examination arrangement request and thereafter extracts an examination resource based on the user information, there are no particular limitations on the order of extraction processing.

Note that the matching unit 122 may perform the matching processing based on not only the examination arrangement request and the user information, but other information as well. For example, the matching unit 122 may perform matching processing based on information such as evaluations or user reviews of examination resources. More specifically, the matching unit 122 may preferentially match the user with an examination resource that has a higher evaluation, or may match the user with an examination resource that has a higher evaluation the higher the priority level (or urgency level, for example) is in the examination arrangement request. Also, the matching unit 122 may perform matching processing based on examination content and examination resources. More specifically, if the content of the examination is a blood test and the examination resource is a blood test kit with a limited period of use (e.g., a blood test kit whose period of use is within 2 days from the date of shipment), the matching unit 122 may match the user with an examination resource that is thought to allow the user to undergo the examination with a sufficient margin based on the usage period. The content of the matching processing performed by the matching unit 122 is not limited to the above description, and can be flexibly changed according to specifications and operations of the medical information processing system.

The output unit 123 is a functional configuration for outputting various types of information to an external device. Also, it should be noted that the matching unit 122 and the output unit 123 described above function as a presentation unit that presents examination resources to the user by performing processing in cooperation with each other. For example, when the user has been matched with an examination resource by the matching unit 122, the output unit 123 outputs the matching processing result to the user terminal 400. At this time, if there are a plurality of matched examination resources, the output unit 123 outputs the examination resources to the user terminal 400 using a predetermined method. For example, the output unit 123 can output the examination resources in order of smallest distance between the user and the examination resource. Also, the output unit 123 may additionally output various types of information regarding examination resources to the user terminal 400 (e.g., the content of examination resources, the dates and times when examination resources can be reserved, the scheduled arrival dates and times of examination resources (if the examination resource is an examination device that can move to the user's position, for example), the scheduled delivery dates and times of examination resource (if the examination resource is a test kit, for example), or a history of examinations undergone by the user in the past). Note that if a history of examinations undergone by the user in the past is to be additionally output, it is desirable that the output unit 123 outputs a predetermined icon for examination resources for which there is a past examination history. It is also desirable that the output unit 123 performs output such that a history of past examinations is displayed when the user selects the icon.

Also, if an arrangement for an examination resource has been made by the management server 200, the output unit 123 outputs the arrangement result to the user terminal 400. If user authentication has been performed by the authentication unit 121, the output unit 123 outputs the result of the user authentication to the user terminal 400. Note that the information output by the output unit 123 and cases where output is performed are not limited to the above examples. Further, the output method used by the output unit 123 can be flexibly changed according to the specifications (or functions, etc.) of the output destination device. For example, the output unit 123 may change the output method according to a mechanism included in the output destination device (e.g., a display mechanism, an audio output mechanism, or a light emitting mechanism).

The storage unit 130 is a functional configuration for storing various types of information. For example, the storage unit 130 stores a table that associates examination resources for use in matching processing with corresponding examination type information. The storage unit 130 also stores information provided by the user terminal 400, the management server 200, or the like (e.g., examination arrangement requests, user information, input information used for user authentication, and examination resource arrangement results), results of processing performed by functional configurations of the matching server 100 (e.g., user authentication results and matching processing results), and the like. The storage unit 130 also stores programs, parameters, and the like used by functional configurations of the matching server 100. Note that the content of the information stored by the storage unit 130 is not limited to these examples.

An example of the functional configurations of the matching server 100 has been described above. Note that the functional configurations described above with reference to FIG. 2 are merely examples, and the functional configurations of the matching server 100 are not limited to these examples. For example, the matching server 100 is not necessarily required to include all of the functional configurations shown in FIG. 2. Also, the functional configurations of the matching server 100 can be flexibly changed according to specifications and operations.

### (Example of Functional Configuration of Management Server 200)

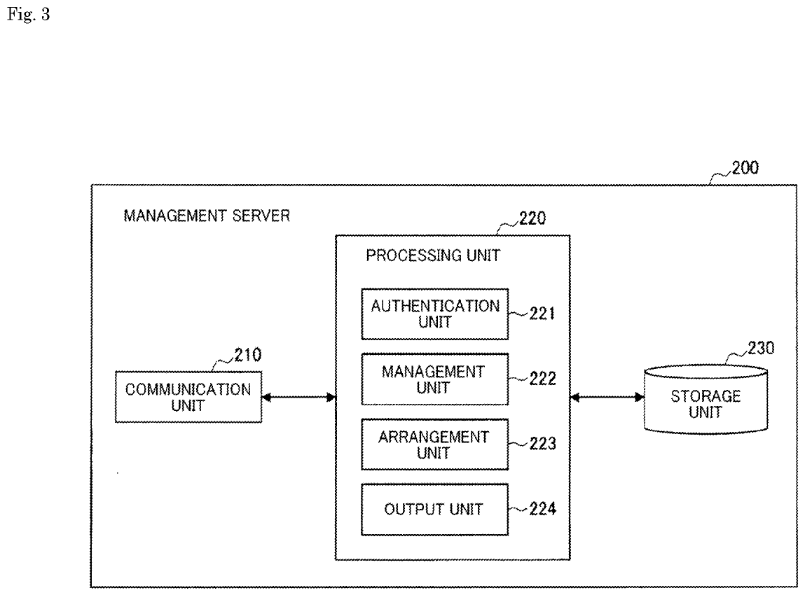

Next, an example of the functional configuration of the management server 200 will be described with reference to FIG. 3. As shown in FIG. 3, the management server 200 includes a communication unit 210, a processing unit 220, and a storage unit 230. The processing unit 220 includes an authentication unit 221, a management unit 222, an arrangement unit 223, and an output unit 224.

The communication unit 210 has a functional configuration for communicating with an external device. For example, the communication unit 210 receives, from the matching server 100, information regarding an examination resource for which an arrangement is to be made. The communication unit 210 also transmits an examination resource arrangement result to the matching server 100. The communication unit 210 also receives, from the user terminal 400, input information regarding examination result output, input information for use in user authentication, and the like. The communication unit 210 also transmits an result of output of the examination result, a user authentication result, and the like to the user terminal 400. The communication unit 210 also receives an examination result and the like from the examination terminal 300. Note that the information transmitted by the communication unit 210 and cases where communication is performed are not limited to the above examples. Also, from the viewpoint of personal information protection, when acquiring the user's personal information or an examination result that includes personal information, the communication unit 210 may be able to determine whether or not consent information indicating whether the user has consented to the acquisition is present, and only acquire such information if the consent information is present.

The processing unit 220 is a functional configuration for performing overall control of all processing performed by the management server 200. For example, the processing unit 220 can control the start and stop of functional configurations. Note that there are no particular limitations on the processing content performed by the processing unit 220. For example, the processing unit 220 may control processing generally performed by various servers, general-purpose computers, PCs, tablet PCs, and the like (e.g., processing related to an OS).

The authentication unit 221 is a functional configuration for performing user authentication. More specifically, there are cases where the user terminal 400 directly accesses the management server 200 without intervention of the matching server 100, and the authentication unit 221 performs user authentication in such cases. Note that the content of the user authentication processing performed by the authentication unit 221 may be similar to that performed by the authentication unit 121 of the matching server 100 described above, and thus will not be described.

The management unit 222 is a functional configuration for managing examination results and examination resources. More specifically, regarding the management of examination results, if an examination result is provided by the examination terminal 300, the management unit 222 stores the examination result in association with user information in the storage unit 230 in a predetermined format. Also, the management unit 222 may delete examination results that are older than a predetermined duration, or write a new examination result over an examination result for the same examination that was performed in the past. Furthermore, if an examination result is provided by the examination terminal 300, the management unit 222 calculates a fee based on the examination content and the like and performs processing for billing the user for the fee. For example, if credit card information or the like has been registered as user information, the management unit 222 performs credit card payment processing or the like based on that information. Also, if the user has selected association with an insurance company, and an examination result has been provided by the examination terminal 300, the management unit 222 may provide a device belonging to the insurance company with an indication that an examination result was provided or the examination result itself. Accordingly, the user can notify the insurance company of the examination frequency and examination results, and therefore can receive a predetermined insurance service (e.g., insurance fee reduction).

More specifically regarding the management of examination resources, if information regarding operation plans for an examination resource (or information regarding the operational status of an examination resource) is provided by the examination resource itself or a device that manages the examination resource (e.g., a server of a medical institution), the management unit 222 stores that information in the storage unit 230 in a predetermined format.

The arrangement unit 223 is a functional configuration for making an arrangement for an examination resource. More specifically, if an examination resource has been determined through a selection made by the user, for example, the arrangement unit 223 makes an examination reservation for the examination resource. For example, the arrangement unit 223 makes an examination reservation by transmitting information indicating a desire for an examination resource arrangement to the examination resource itself or a predetermined device of the examination resource (e.g., a server of an medical institution that is the examination resource).

The “information indicating a desire for an examination resource arrangement” includes, for example, user information (e.g., attribute information) regarding the user who is to undergo an examination, user identification information, user position information, examination date/time, examination location (if the examination resource is an examination device that can move to the user's position, for example), and examination content information. Note that the information included in the information indicating a desire for an examination resource arrangement is not limited to these examples. Also, such information may be set based on user input, or may be set automatically by a function of the user terminal 400 or the management server 200. Moreover, if personal information such as user information is included in the information indicating a desire for an examination resource arrangement, it is desirable that processing for obtaining consent from the user is performed in advance.

The output unit 224 is a functional configuration for outputting various types of information to an external device. For example, if an examination resource arrangement has been made by the arrangement unit 223, the output unit 224 outputs the examination resource arrangement result to the matching server 100. Also, if user authentication has been performed by the authentication unit 221, the output unit 224 outputs the result of the user authentication to the user terminal 400.

The output unit 224 also outputs an examination result to a user-desired device based on direct or indirect input performed by the user. For example, the output unit 224 outputs an examination result to a device at an institution where a diagnosis, treatment, surgery, or estimation of user symptoms is to be performed based on the examination result. Here, “direct input performed by the user” includes the case where the user chooses to download the examination result (save the examination result to the user terminal 400), for example. Also, “indirect input performed by the user” includes the case where the user receives a request from a diagnostic service provider (including a diagnostic service providing application for estimating symptoms through machine learning, for example) and chooses to upload the examination result (save the examination result to a server of the diagnostic service provider). Note that the output unit 224 may additionally output information other than the examination result (e.g., user information). Also, the output unit 224 may control the information that is to be output in accordance with the output destination. For example, if the output destination is a device other than the user terminal 400, the output unit 224 may not output personal information such as user information. Also, from the viewpoint of personal information protection, in the case where the user desires to upload an examination result to a diagnostic service provider, it is preferable that the output unit 224 performs consent processing to allow the user to select whether or not to consent to the uploading of the examination result, so as to generate consent information that indicates that the user consented to the uploading of the examination result, and then provide the consent information to the diagnostic service provider.

Also, if an examination result has been output to a user-desired device, the output unit 224 outputs the result of output to the user terminal 400. Note that the information output by the output unit 224 and cases where output is performed are not limited to the above examples. Further, the output method used by the output unit 224 can be flexibly changed according to the specifications (or functions, etc.) of the output destination device. For example, the output unit 224 may change the output method according to a mechanism included in the output destination device (e.g., a display mechanism, an audio output mechanism, or a light emitting mechanism).

The storage unit 230 is a functional configuration for storing various types of information. For example, the storage unit 230 stores examination results associated with user information, and information regarding operation plans for examination resources (or information regarding the operational statuses of examination resources) in a predetermined format. The storage unit 230 also stores information provided by the matching server 100, the examination terminal 300, the user terminal 400, and the like (e.g., input information for use in user authentication), the results of processing performed by functional configurations of the management server 200 (e.g., user authentication results and examination resource arrangement results), and the like. The storage unit 230 also stores programs, parameters, and the like used by functional configurations of the management server 200. Note that the content of the information stored by the storage unit 230 is not limited to these examples.

An example of the functional configurations of the management server 200 has been described above. Note that the functional configurations described above with reference to FIG. 3 are merely examples, and the functional configurations of the management server 200 are not limited to these examples. For example, the management server 200 is not necessarily required to include all of the functional configurations shown in FIG. 3. Also, the functional configurations of the management server 200 can be flexibly changed according to specifications and operations.

### (Example of Functional Configuration of User Terminal 400)

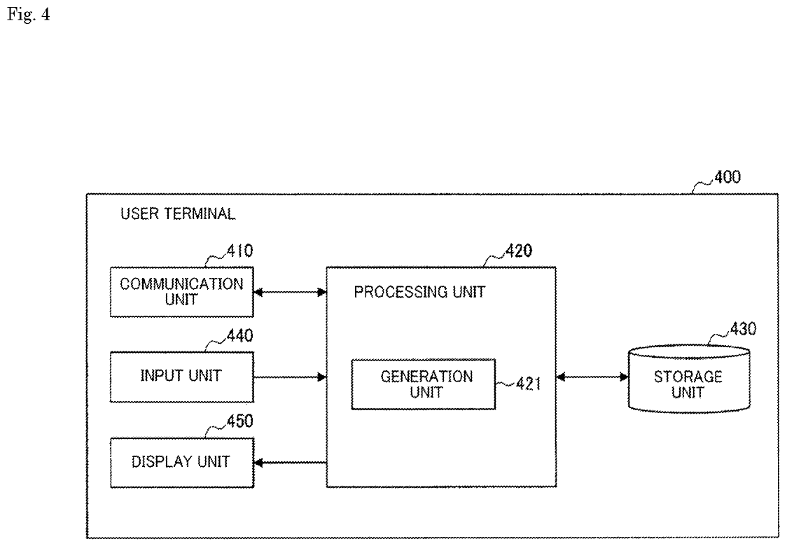

Next, an example of the functional configuration of the user terminal 400 will be described with reference to FIG. 4. As shown in FIG. 4, the user terminal 400 includes a communication unit 410, a processing unit 420, a storage unit 430, an input unit 440, and a display unit 450. Also, the processing unit 420 includes a generation unit 421.

The communication unit 410 has a functional configuration for communicating with an external device. For example, the communication unit 410 transmits an examination arrangement request, user information, input information for use in user authentication, and the like to the matching server 100. The communication unit 410 also receives a result of examination resource matching processing, a result of examination resource arrangement processing, a user authentication result, and the like from the matching server 100. The communication unit 410 also transmits input information regarding examination result output, input information for use in user authentication, and the like to the management server 200. The communication unit 410 also receives an examination resource output result, a user authentication result, and the like from the management server 200. Note that the information transmitted by the communication unit 410 and cases where communication is performed are not limited to the above examples.

The processing unit 420 is a functional configuration for performing overall control of all processing performed by the user terminal 400. For example, the processing unit 420 can control the start and stop of functional configurations. Note that there are no particular limitations on the processing content performed by the processing unit 420. For example, the processing unit 420 may control processing generally performed by various servers, general-purpose computers, PCs, tablet PCs, and the like (e.g., processing related to an OS).

The generation unit 421 is a functional configuration that performs processing regarding to a medical inquiry application and generates an examination arrangement request. Here, “medical inquiry application” refers to an application for performing a medical inquiry in the place of a doctor or the like, and is installed in the user terminal 400 (or in another device that cooperates with the user terminal 400 as necessary). Note that the medical inquiry application analyzes input performed by the user to perform a medical inquiry and generate an examination arrangement request, and realizes such processing with use of a predetermined AI (Artificial Intelligence). More specifically, the generation unit 421 inputs information input by the user (e.g., text information or still image information or video information that shows an affected part) to the AI of the medical inquiry application, and receives a medical inquiry result and an examination arrangement request. The functionality of the AI can be realized based on a machine learning technique such as a neural network or a regression model, or based on a statistical technique, for example. Hereinafter, the medical inquiry application of the present embodiment will sometimes be called the “medical inquiry AI”. The functions of the medical inquiry AI and a specific example of a user interface will be described later. Note that the means for realizing the functions of the medical inquiry application may be a technique such as a decision tree or a random forest, linear regression, or a Bayesian model.

The storage unit 430 is a functional configuration for storing various types of information. For example, the storage unit 430 stores information provided by the matching server 100, the management server 200, and the like (e.g., matching processing results, examination resource arrangement results, user authentication results, and examination resource output results), results of processing performed by functional configurations of the user terminal 400 (e.g., medical inquiry results and examination arrangement requests), and the like. The storage unit 430 also stores programs, parameters, and the like used by functional configurations of the user terminal 400. Note that the content of the information stored by the storage unit 430 is not limited to these examples.

The input unit 440 is a functional configuration for receiving user input. For example, the input unit 440 includes input devices such as a mouse, a keyboard, a touch panel, a button, a switch, a microphone, or a camera, and the user can input desired information by using such input devices. Further, the input unit 440 is provided with various types of sensor devices such as a position sensor, an acceleration sensor, or a gyro sensor, and the user's position information (e.g., latitude, longitude, and altitude) can be input by the position sensor, for example. Note that there are no particular limitations on the input devices and the sensor devices that are included in the input unit 440.

The display unit 450 is a functional configuration for displaying various types of information. More specifically, the display unit 450 includes a display device such as a display, a projection device such as a projector, or the like, and by using these devices, a processing result of the device, information output by the matching server 100 and the management server 200, and the like can be provided to the user. Note that there are no particular limitations on the devices that are included in the display unit 450.

An example of the functional configurations of the user terminal 400 has been described above. Note that the functional configurations described above with reference to FIG. 4 are merely examples, and the functional configurations of the user terminal 400 are not limited to these examples. For example, the user terminal 400 is not necessarily required to include all of the functional configurations shown in FIG. 4. Also, the functional configurations of the user terminal 400 can be flexibly changed according to specifications and operations.

### (1.3. Processing Flow)

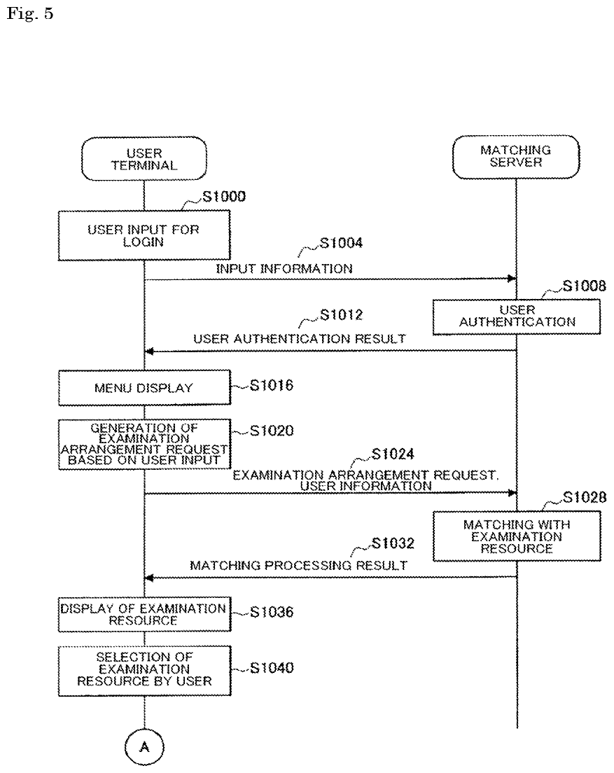

Examples of the functional configurations of devices included in the medical information processing system have been described above. Next, an example of the flow of processing performed by the devices will be described with reference to FIGS. 5 to 7.

### (Example of Flow of Processing Up to Examination Resource Arrangement)

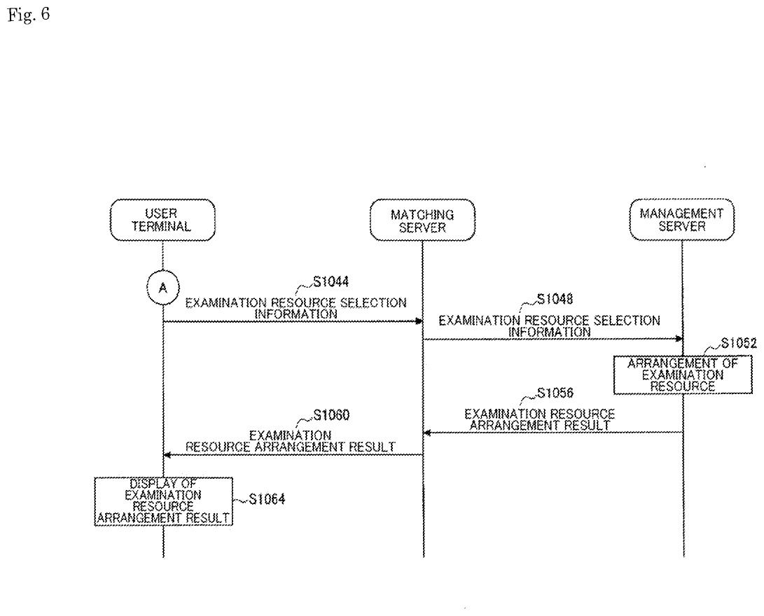

First, an example of the flow of processing up to the arrangement of an examination resource will be described with reference to FIGS. 5 and 6.

In step S1000, the user uses the input unit 440 of the user terminal 400 to perform input for logging into the medical information processing system. For example, the user inputs user identification information (e.g., an ID) and a password, or input biometric information for biometric authentication. Note that input operations for login may be automated using a function of the user terminal 400. In step S1004, the communication unit 410 transmits the input information input by the user to the matching server 100. For example, the communication unit 410 transmits hash path information, in which the user identification information (e.g., an ID) and the password are hashed, as the input information.

In step S1008, the authentication unit 121 of the matching server 100 performs predetermined user authentication processing using the input information. For example, the authentication unit 121 performs user authentication based on whether or not the hash path information provided as input information matches hash path information that has been registered in advance. In step S1012, the output unit 123 outputs the user authentication result to the user terminal 400 via the communication unit 110.

If the user authentication was successful, in step S1016, the display unit 450 of the user terminal 400 displays a medical inquiry AI menu. In step S1020, the generation unit 421 generates an examination arrangement request based on user input. In step S1024, the communication unit 410 transmits the examination arrangement request and the user information to the matching server 100.

In step S1028, the matching unit 122 of the matching server 100 performs matching between the user and examination resources based on the examination arrangement request and the user information. In step S1032, the output unit 123 outputs the matching processing result to the user terminal 400 via the communication unit 110. For example, if two or more examination resources were extracted as the matching processing, the output unit 123 outputs information regarding each of the candidate examination resources to the user terminal 400.

In step S1036, the display unit 450 of the user terminal 400 displays the examination resources that are the matching processing result. In step S1040, the user selects an examination resource using the input unit 440. Note that if only one examination resource is provided as the matching processing result, the user uses the input unit 440 to select whether or not to undergo an examination through the examination resource.

In step S1044, the communication unit 410 transmits information regarding the examination resource selected by the user (in the figure, shown as “examination resource selection information”) to the matching server 100. In step S1048, the output unit 123 of the matching server 100 outputs the examination resource selection information to the management server 200 via the communication unit 110.

In step S1052, the arrangement unit 223 of the management server 200 makes an examination resource arrangement based on the examination resource selection information. In step S1056, the output unit 224 outputs the examination resource arrangement result to the matching server 100 via the communication unit 210. in step S1060, the output unit 123 of the matching server 100 outputs the examination resource arrangement result to the user terminal 400 via the communication unit 110. In step S1064, the display unit 450 of the user terminal 400 displays the examination resource arrangement result, and then the series of processing up to examination resource arrangement ends.

### (Example of Flow of Examination Result Output Processing)

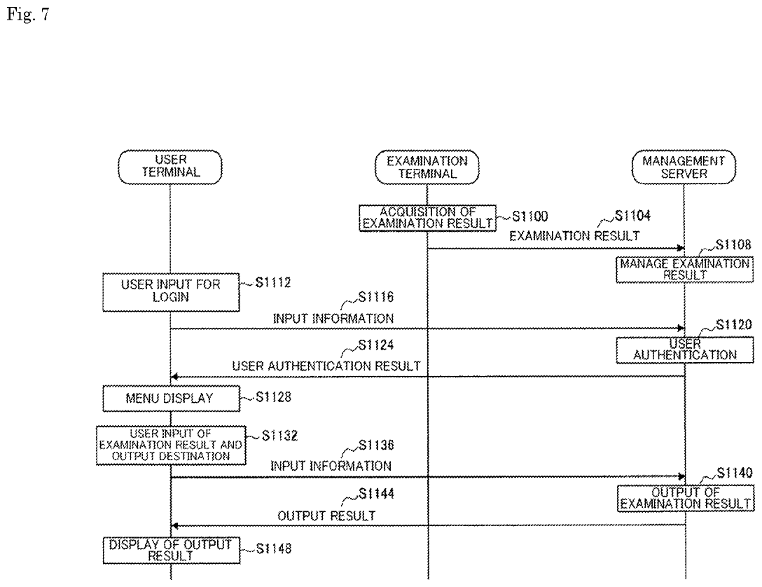

Next, an example of the flow of examination result output processing will be described with reference to FIG. 7.

In step S1100, after an examination has ended, the examination terminal 300 acquires an examination result. In step S1104, the examination terminal 300 transmits the examination result to the management server 200. In step S1108, the management unit 222 of the management server 200 manages the examination result by storing the examination result in association with user information in the storage unit 230 in a predetermined format.

Thereafter, if the user desires to take any of various actions based on the examination result (e.g., uses a diagnostic service), in step S1112, the user uses the input unit 440 of the user terminal 400 to perform input for logging into the medical information processing system. In step S1116, the communication unit 410 transmits the input information input by the user to the management server 200.

In step S1120, the authentication unit 221 of the management server 200 performs predetermined user authentication processing using the input information. In step S1124, the output unit 224 outputs a user authentication result to the user terminal 400 via the communication unit 210.

If the user authentication was successful, in step S1128, the display unit 450 of the user terminal 400 displays a predetermined menu. Note that the menu display may be realized by the medical inquiry AI. In step S1132, the user uses the input unit 440 to input an examination result that is to be output and an output destination.

In step S1136, the communication unit 410 transmits the input information input by the user to the management server 200. In step S1140, the output unit 224 of the management server 200 outputs the examination result to the user-desired device based on the input information. In step S1144, the output unit 224 outputs the examination resource output result to the user terminal 400 via the communication unit 210. In step S1148, the display unit 450 of the user terminal 400 displays the examination resource output result, and then the series of examination result output processing ends.

Note that the steps in the sequence diagrams of FIGS. 5 to 7 do not necessarily need to be performed in a time series that follows the above-described order. In other words, the steps in the sequence diagrams may be performed in an order different from the above-described order, or may be performed in parallel.

### 2. Second Embodiment

### (2.1. System Configuration)

The first embodiment of the present disclosure has been described above. The following describes a second embodiment of the present disclosure.

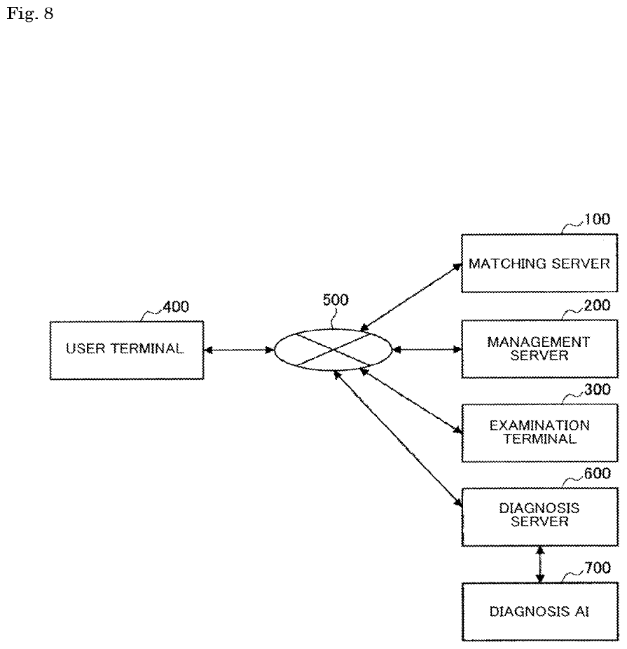

First, an example of the system configuration of a medical information processing system according to the second embodiment of the present disclosure will be described with reference to FIG. 8. As shown in FIG. 8, the medical information processing system of the present embodiment further includes a diagnosis server 600 and a diagnosis AI 700. Note that the other devices may be the same as those in the first embodiment, and thus will not be described.

### (Diagnosis Server 600)

The diagnosis server 600 is a medical information processing device for controlling user symptom estimation processing performed using the diagnosis AI 700. More specifically, if the management server 200 has output an examination result to the diagnosis server 600 based on user input, the diagnosis server 600 inputs the examination result to the diagnosis AI 700 and receives symptom estimation result output.

Then, using identification information of the examination result (e.g., an examination ID), the diagnosis server 600 generates comprehensive result information by associating the estimation result with the examination result.

The diagnosis server 600 then provides the comprehensive result information to a predetermined device (e.g., the user terminal 400). Accordingly, the user can acquire a symptom estimation result.

### (Diagnosis AI 700)

The diagnosis AI 700 is a functional configuration that functions as an estimation unit for performing user symptom estimation based on an examination result provided by the diagnosis server 600 (note that the diagnosis server 600 that uses the diagnosis AI 700 may function as the estimation unit). More specifically, the diagnosis AI 700 is an application that is installed in a predetermined medical information processing device, and performs user symptom estimation using AI. Also, the diagnosis AI 700 may additionally make a proposal to undergo a medical consultation at a medical institution, take a medication, or the like based on the user symptom estimation result.

The functionality of the AI used in the diagnosis AI 700 (or the medical inquiry AI) may be realized based on a machine learning technique such as a neural network or a regression model, or based on a statistical technique, for example. For example, in the case of using a machine learning technique, the functionality of the AI is realized by a processing circuit that includes a processing model whose parameters were generated by training in which training data that includes doctor-provided diagnosis results in association with examination results, or training data that includes medical inquiry results in association with medical inquiry content is input to a predetermined calculation model that uses a neural network or a regression model. Note that the diagnosis AI 700 may directly acquire the examination result from the management server 200 without intervention of the diagnosis server 600. Also, the diagnosis AI 700 may be provided in the diagnosis server 600, or may be an application equivalent to the medical inquiry AI described above (e.g., the diagnosis AI 700 may be an application that is equivalent to the medical inquiry AI and is provided in the user terminal 400).

### (2.2. Functional Configuration of Device)

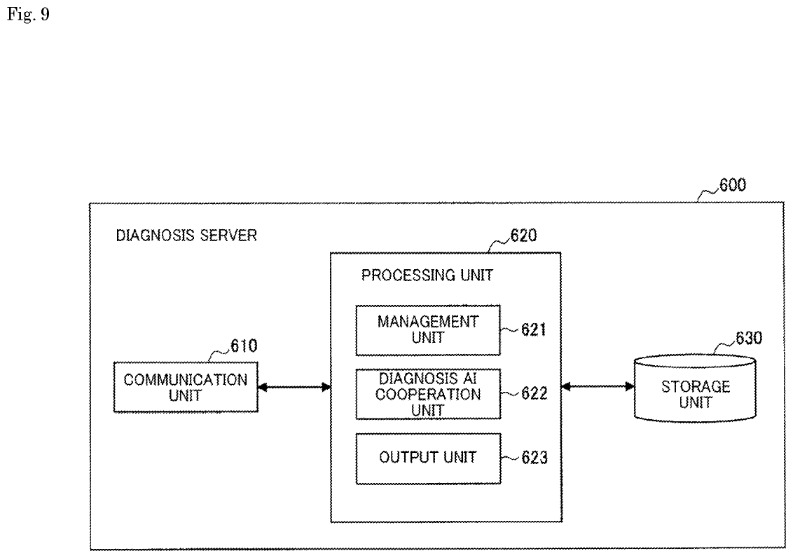

An example of the system configuration of the medical information processing system according to the second embodiment has been described above. Next, an example of the functional configuration of the diagnosis server 600 of the present embodiment will be described with reference to FIG. 9. Note that the functional configurations of the other devices may be the same as those in the first embodiment, and thus will not be described.

As shown in FIG. 9, the diagnosis server 600 includes a communication unit 610, a processing unit 620, and a storage unit 630. Further, the processing unit 620 includes a management unit 621, a diagnosis AI cooperation unit 622, and an output unit 623.

The communication unit 610 has a functional configuration for communicating with an external device. For example, the communication unit 610 receives an examination result or the like from the management server 200. The communication unit 610 then transmits the examination result to the diagnosis AI 700 and receives a user symptom estimation result from the diagnosis AI 700. Thereafter, the communication unit 610 transmits a comprehensive result information, which was generated using the estimation result, to the user terminal 400, for example. Note that the information transmitted by the communication unit 610 and cases where communication is performed are not limited to the above examples.

The processing unit 620 is a functional configuration for performing overall control of all processing performed by the diagnosis server 600. For example, the processing unit 620 can control the start and stop of functional configurations. Note that there are no particular limitations on the processing content performed by the processing unit 620. For example, the processing unit 620 may control processing generally performed by various servers, general-purpose computers, PCs, tablet PCs, and the like (e.g., processing related to an OS (Operating System)).

The management unit 621 is a functional configuration for managing comprehensive result information. More specifically, if an user symptom estimation result was provided by the diagnosis AI 700, the management unit 621 generates a comprehensive result information by associating the estimation result with an examination result with use of identification information of the examination result (e.g., an examination ID). At this time, the management unit 621 may also include various types of information regarding the diagnosis AI 700 and the diagnosis (e.g., the name, type, and version information of the diagnosis AI 700, and the date/time when the diagnosis was made) in the comprehensive result information. In other words, the management unit 621 can perform management such that it is possible to recognize the diagnosis AI 700 and the examination result that served as the basis for obtaining the user symptom estimation result.

The management unit 621 then stores the comprehensive result information in the storage unit 630 in a predetermined format. Also, the management unit 621 may delete comprehensive result information that is older than a predetermined duration, or write new comprehensive result information over comprehensive result information for the same examination that was performed in the past. Furthermore, if a user symptom estimation result is provided by the diagnosis AI 700, the management unit 621 calculates a fee based on the estimation result and the like and performs processing for billing the user for the fee. For example, if credit card information or the like has been registered as user information, the management unit 621 performs credit card payment processing or the like based on that information. Also, if the user has selected association with an insurance company, and a user symptom estimation has been provided by the diagnosis AI 700, the management unit 621 may provide a device belonging to the insurance company with an indication that a user symptom estimation result was provided or the comprehensive result information. Accordingly, the user can notify the insurance company of the diagnosis frequency and comprehensive result information, and therefore can receive a predetermined insurance service (e.g., insurance fee reduction).

The diagnosis AI cooperation unit 622 is a functional configuration for performing cooperation with the diagnosis AI 700 in the user symptom estimation processing. More specifically, if an examination result was provided by the management server 200, the diagnosis AI cooperation unit 622 inputs the examination result to the diagnosis AI 700 via the communication unit 610. Thereafter, when user symptom estimation processing performed by the diagnosis AI 700 has ended, the diagnosis AI cooperation unit 622 receives a user symptom estimation result via the communication unit 610. Note that if there are two or more diagnosis AIs 700, the diagnosis AI cooperation unit 622 may select the diagnosis AI 700 that is to be used for symptom estimation processing. For example, the diagnosis AI cooperation unit 622 may select the diagnosis AI 700 that is to be used for symptom estimation processing based on information such as examination or examination resource content, examination priority level (or urgency level, for example), examination result, historical information regarding a diagnosis AI 700 that was used in the past for the same user, setting information regarding the diagnosis AI 700 that is to be used in the future (e.g., the user's requests, needs, or restrictions regarding the diagnosis AI 700), evaluations or user reviews of a diagnosis AI 700, and the like. Note that the method by which the diagnosis AI cooperation unit 622 selects a diagnosis AI 700 is not limited to these examples, and may be flexibly changed according to specifications and operations of the medical information processing system.

The output unit 623 is a functional configuration for outputting various types of information to an external device. For example, if comprehensive result information was generated by the management unit 621, the output unit 623 outputs the comprehensive result information to the user terminal 400 or the like. Note that the output destination of the comprehensive result information is not limited to being the user terminal 400. For example, the output unit 623 may output the comprehensive result information to the management server 200 so as to provide the comprehensive result information to the user via the management server 200. Further, the output method used by the output unit 623 can be flexibly changed according to the specifications (or functions, etc.) of the output destination device. For example, the output unit 623 may change the output method according to a mechanism included in the output destination device (e.g., a display mechanism, an audio output mechanism, or a light emitting mechanism).

The storage unit 630 is a functional configuration for storing various types of information. For example, the storage unit 630 stores examination results, user symptom estimation results, and the like. The storage unit 630 also stores programs, parameters, and the like used by functional configurations of the diagnosis server 600. Note that the content of the information stored by the storage unit 630 is not limited to these examples.

An example of the functional configurations of the diagnosis server 600 has been described above. Note that the functional configurations described above with reference to FIG. 9 are merely examples, and the functional configurations of the diagnosis server 600 are not limited to these examples. For example, the diagnosis server 600 is not necessarily required to include all of the functional configurations shown in FIG. 9. Also, in the case where the diagnosis AI 700 also makes a proposal to undergo a medical consultation at a medical institution or to take a medication, for example, the diagnosis server 600 may include a function for making an arrangement for a medical consultation at an medical institution or for a medication (at this time, the function may be the same as or similar to the function provided in the arrangement unit 223 of the management server 200). Also, the functional configurations of the diagnosis server 600 can be flexibly changed according to specifications and operations.

### (2.3. Processing Flow)

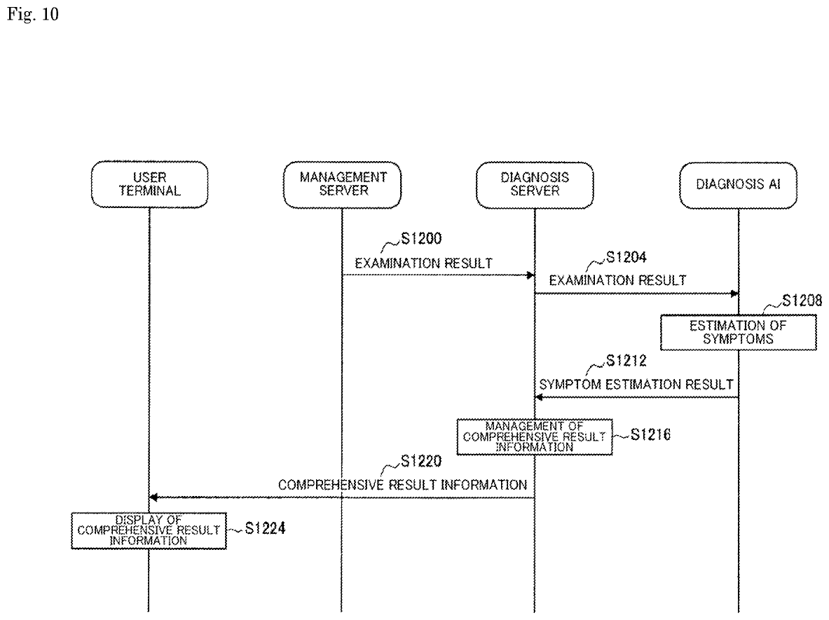

An example of the functional configurations of the diagnosis server 600 of the present embodiment has been described above. Next, an example of the flow of processing of the present embodiment will be described with reference to FIG. 10. FIG. 10 is a sequence diagram showing an example of the flow of processing related to the provision of comprehensive result information to a user.

In step S1200, the output unit 224 of the management server 200 outputs an examination result to the diagnosis server 600 via the communication unit 210. In step S1204, the diagnosis AI cooperation unit 622 of the diagnosis server 600 inputs the examination result to the diagnosis AI 700 via the communication unit 610.

In step S1208, the diagnosis AI 700 performs user symptom estimation using the input examination result. In step S1212, the diagnosis AI cooperation unit 622 of the diagnosis server 600 receives a user symptom estimation result from the diagnosis AI 700 via the communication unit 610. In step S1216, the management unit 621 generates comprehensive result information by associating the estimation result with the examination result based on identification information of the examination result (e.g., an examination ID), and manages the comprehensive result information.

In step S1220, the output unit 623 outputs the comprehensive result information to the user terminal 400 via the communication unit 610. In step S1224, the display unit 450 of the user terminal 400 displays the comprehensive result information, and then the series of processing regarding the provision of comprehensive result information ends.

Note that the steps in the sequence diagram of FIG. 10 do not necessarily need to be performed in a time series that follows the above-described order. In other words, the steps in the sequence diagram may be performed in an order different from the above-described order, or may be performed in parallel.

### 3. User Interface

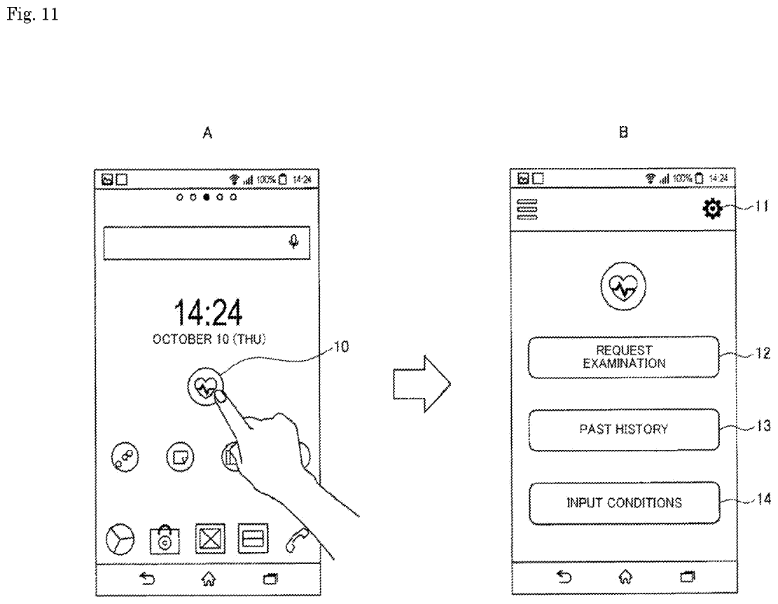

The second embodiment of the present disclosure has been described above. Next, a specific example of a medical inquiry AI user interface will be described with reference to FIGS. 11 to 17. As described above, “medical inquiry AI” refers to an application for performing a medical inquiry in the place of a doctor or the like, and is for performing a medical inquiry by analyzing user input, and generating an examination arrangement request. The following describes various types of user interfaces, including some for the generation of an examination arrangement request. Note that the following describes an example of the case where the user terminal 400 in which the medical inquiry AI is installed is a smartphone (of course, the user terminal 400 is not limited to being a smartphone).

First, the user can launch the medical inquiry AI by selecting an icon 10 shown under A in FIG. 11 by a method such as tapping. When the medical inquiry AI has been launched, a menu screen is displayed as shown under B in FIG. 11.

A button 11 displayed in the menu screen is a button used for moving to a user information setting screen. In the screen moved to by selection of the button 11, the user can set, as user information, user attribute information (e.g., name, gender, age, blood type, address, telephone number, family structure, occupation, insurance information, and credit card information), setting information regarding an examination that the user is to undergo in the future (e.g., the user's requests, needs, or restrictions regarding the examination), and the like.

An examination request button 12 is a button used when requesting an arrangement for an examination resource. A past history button 13 is a button used when checking historical information regarding past examination requests (i.e., requests for arrangement of an examination resource) or past examination results. A condition input button 14 is a button used when inputting the user's current condition (e.g., height, weight, body fat percentage, BMI, temperature, visual acuity, and auditory acuity). Note that the user's current condition may be input through cooperation with an external device such as a wearable device.

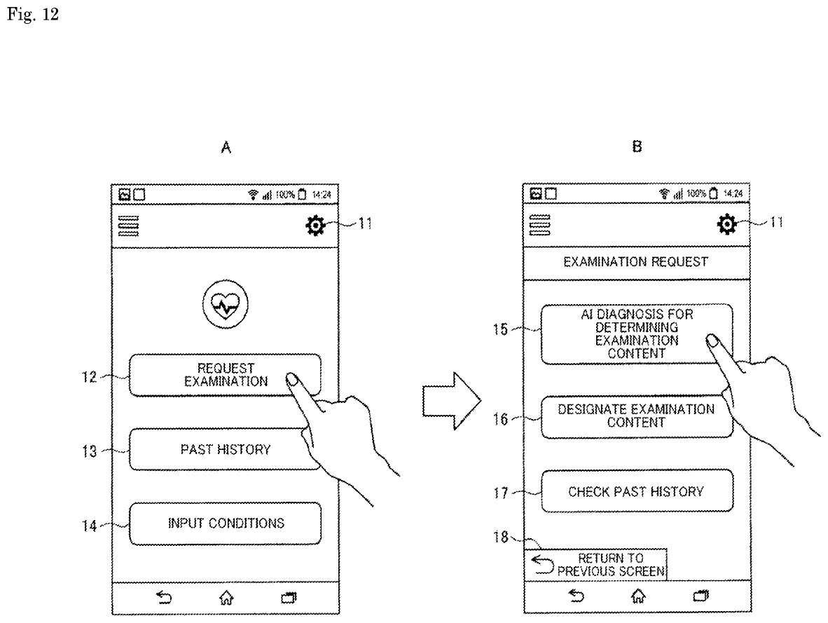

The user can move to an examination request screen shown under B in FIG. 12 by selecting the examination request button 12 shown under A in FIG. 12 (the same screen as in B of FIG. 11) by a method such as tapping. A button 15 is a button used when selecting a desired examination type from among examination types recommended by the medical inquiry AI after the user has undergone a medical inquiry performed by the medical inquiry AI (hereinafter, called an “AI medical inquiry”). A button 16 is a button used in the case where the user designates an examination type without undergoing an AI medical inquiry. A button 17 is a button used in the case where the user checks a type of examination that the user underwent in the past, and selects the same examination type. In this way, multiple examination request methods are provided, thus making it possible for the user to easily determine an examination type and make an examination request. A button 18 is a button used when moving to the menu screen shown under A in FIG. 12.

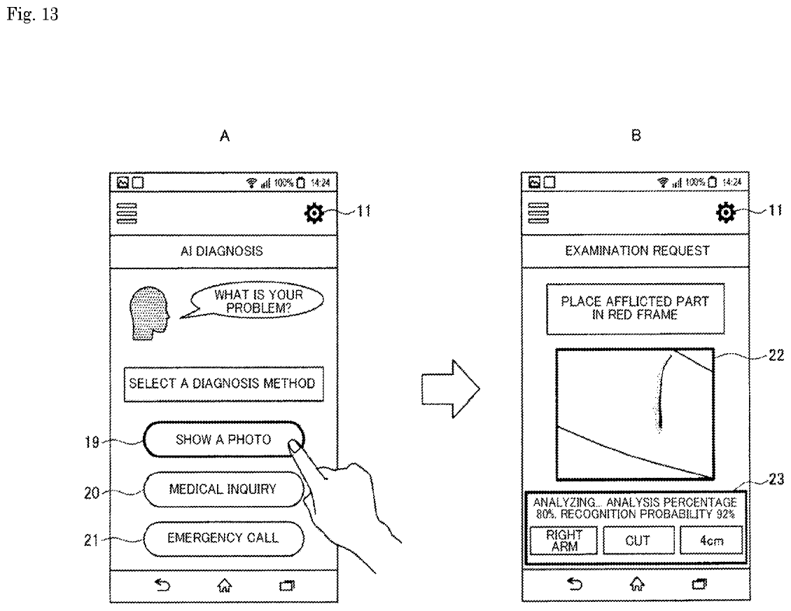

The screen shown under A in FIG. 13 is an AI medical inquiry menu screen that is displayed after the user has selected the button 15 as shown under B in FIG. 12. A button 19 is a button used in the case where the user undergoes an AI medical inquiry based on image information (e.g., still image information or video information that shows an affected part). A button 20 is a button used in the case where the user undergoes a medical inquiry by answering questions given by the medical inquiry AI. A button 21 is a button used in the case where the user undergoes a medical inquiry by calling a medical examination worker such as a doctor. In this way, multiple medical inquiry methods are provided, thus making it possible for the user to undergo a medical inquiry that is appropriate for the user's symptoms.

The user can move to an image information input screen shown under B in FIG. 13 by selecting a button 19 shown under A in FIG. 13 by a method such as tapping. An area 22 is an area for displaying captured image information generated by a camera included in the user terminal 400, for example. The user checks the area 22 and presses a predetermined button so as to input captured image information that shows an affected part. Note that if the user terminal 400 is a device that is held in one hand by the user when performing image capturing, such as a smartphone, it is thought to be easier for the user to perform image capturing when the captured image information is video information as compared with still image information, and therefore it is desirable that the captured image information is video information. An area 23 is an area that displays a result of an analysis of the captured image information by the medical inquiry AI. More specifically, the medical inquiry AI analyzes the input captured image information in order to estimate the location, condition, or severity of the affected part, and also outputs the analysis progress, estimated accuracy of the analysis result, and the like. In the example shown under B in FIG. 13, the displayed analysis result includes an indication that there is a 4 cm cut (condition and severity of the affected part) on the user's right arm (location of affected part), and an indication that the analysis percentage is 80% (analysis progress) and the recognition probability is 92% (estimated accuracy of the analysis result).

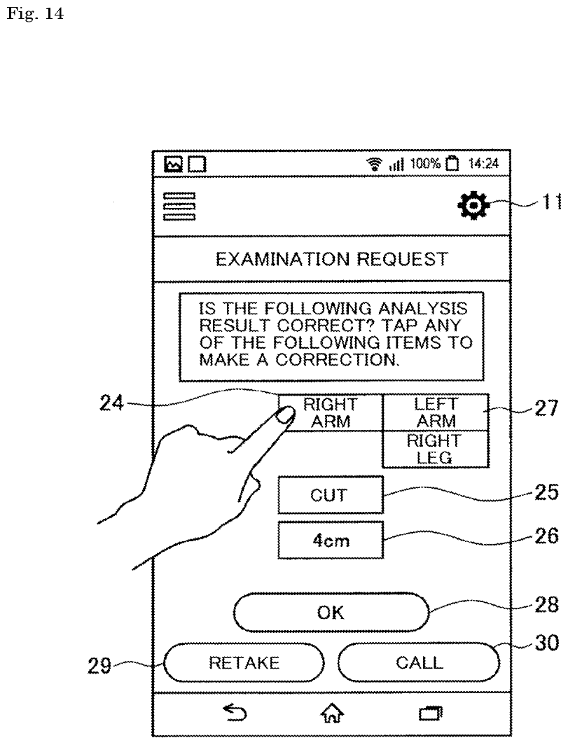

The screen shown in FIG. 14 is a screen moved to after captured image information was input and an analysis ended in the screen shown under B in FIG. 13. Buttons 24 to 26 are buttons used when correcting the analysis result displayed in the area 23 under B in FIG. 13. For example, if the user selects the button 24, a pulldown list 27 showing correction candidates is displayed. For example, the pulldown list 27 displays candidates output by the analysis of the captured image information performed by the medical inquiry AI (in the example in FIG. 14, “left arm” and “right leg” are displayed as correction candidates). Accordingly, even if the analysis accuracy of the medical inquiry AI is low, the user can appropriately correct the analysis result. An OK button 28 is a button used when the user checks and corrects the analysis result and then finalizes the content. A retake button 29 is a button used when returning to the screen shown under B in FIG. 13 and performing the input of captured image information again. A call button 30 is a button used when calling a help desk to check how to perform operations, for example.

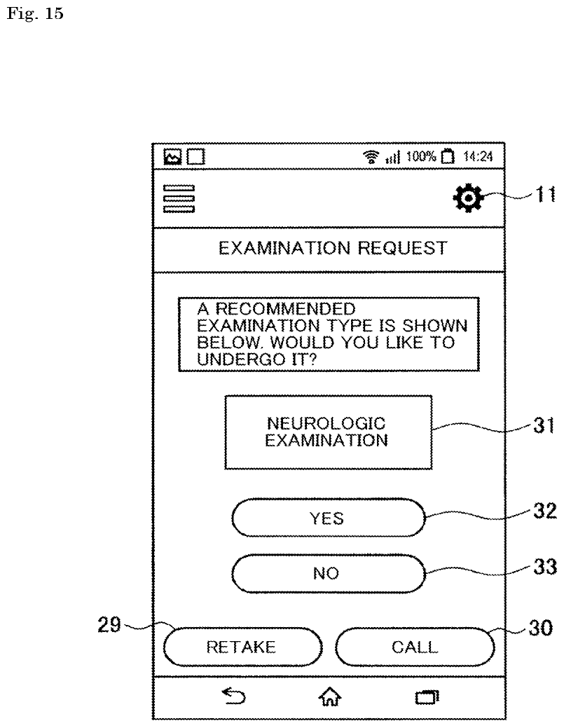

The screen shown in FIG. 15 is a screen moved to when the OK button 28 in the screen shown in FIG. 14 is selected by a method such as tapping. The medical inquiry AI outputs a recommended examination type based on the result (or the corrected content) of the captured image information analysis (neurologic examination in the example in FIG. 15). A button 32 is a button used when the user desires to undergo an examination of the recommended examination type. If the user selects the button 32, the medical inquiry AI generates an examination arrangement request, the examination arrangement request is transmitted to the matching server 100, and matching processing is performed. A button 33 is a button used if the user does not desire to undergo an examination of the recommended examination type. If the user selects the button 33, examination arrangement request generation and matching processing will not be performed, and text information indicating “It is recommended to undergo a medical consultation at a medical institution as soon as possible”, for example, is displayed.

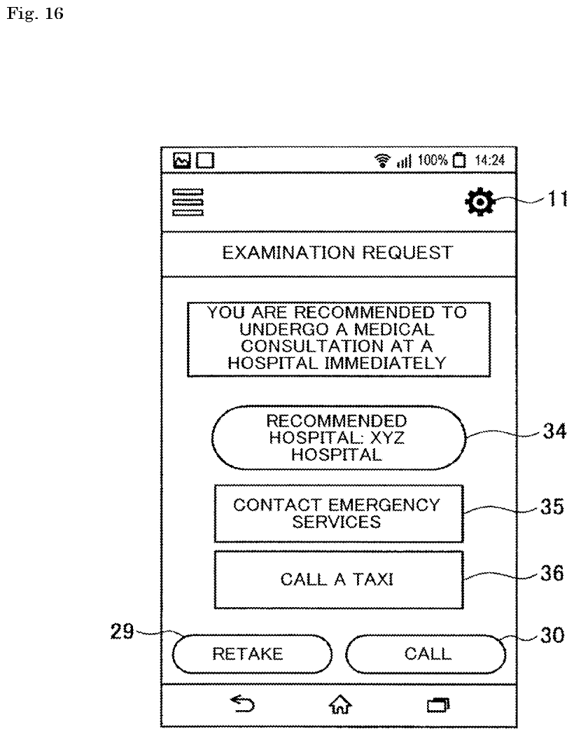

The screen shown in FIG. 16 is a screen moved to when the OK button 28 in the screen shown in FIG. 14 is selected by a method such as tapping, and is displayed in the case where the medical inquiry AI determined that the level of urgency is high. An area 34 is an area for displaying a medical institution recommended by the medical inquiry AI. The medical inquiry AI outputs a medical institution that can perform an examination based on the medical inquiry result and the user information (e.g., the user's position information) and that can be accessed by the user in a short time, and displays the medical institution in the area 34. Note that the matching server 100 may provide the recommended medical institution by cooperating with the medical inquiry AI, and the medical inquiry AI may display that medical institution in the area 34. A button 35 is a button used when requesting emergency medical care, and a button 36 is a button used when requesting a taxi dispatch. Due to these buttons being provided, the user can quickly and easily obtain access to the recommended medical institution.

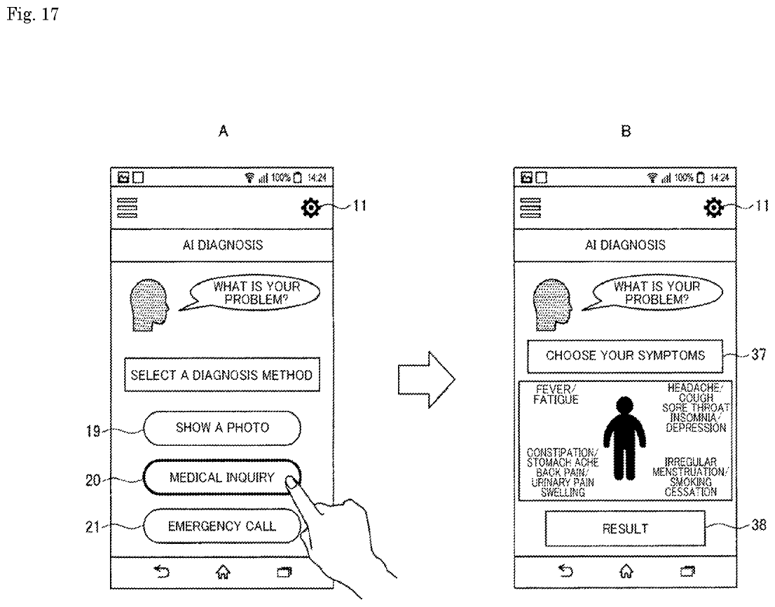

If the user has selected the button 20 in the above-described screen under A in FIG. 13 (the same screen as under A in FIG. 17), the screen shown under B in FIG. 17 is displayed. A question from the medical inquiry AI is displayed in an area 37 (in the example under B in FIG. 17, the message “Select your symptoms” is displayed, and the user can undergo a medical inquiry by answering such questions. A result button 38 is a button displayed when the last question has been answered, and is for moving to a screen that displays the medical inquiry result. If the result button 38 was selected, the screen in FIG. 15 (the screen in FIG. 16 if the level of urgency is high) is displayed for example (at this time, the retake button 29 and the like are omitted as appropriate). According, an examination arrangement request is appropriately generated based on the answers to the questions from the medical inquiry AI as well (rather than captured image information). For example, if the medical inquiry AI inferred based on the user's answers that the symptoms indicate influenza, and the user desires to undergo an examination, the medical inquiry AI extracts a hospital (a hospital that can perform an influenza examination) that is close to the user from among the examination resources based on the user's position information.

Next, the medical inquiry AI presents time slots of availability at the hospital to the user. When the user designates a time slot, the medical inquiry AI generates an examination arrangement request based on the estimated symptoms and selected time slot, and transmits the examination arrangement request. At this time, it is preferable that the medical inquiry AI accesses a reservation database of the hospital, acquires time slots when a reservation can be made, and presents the reservable time slots as time slots of hospital availability.

### 4. Hardware Configuration

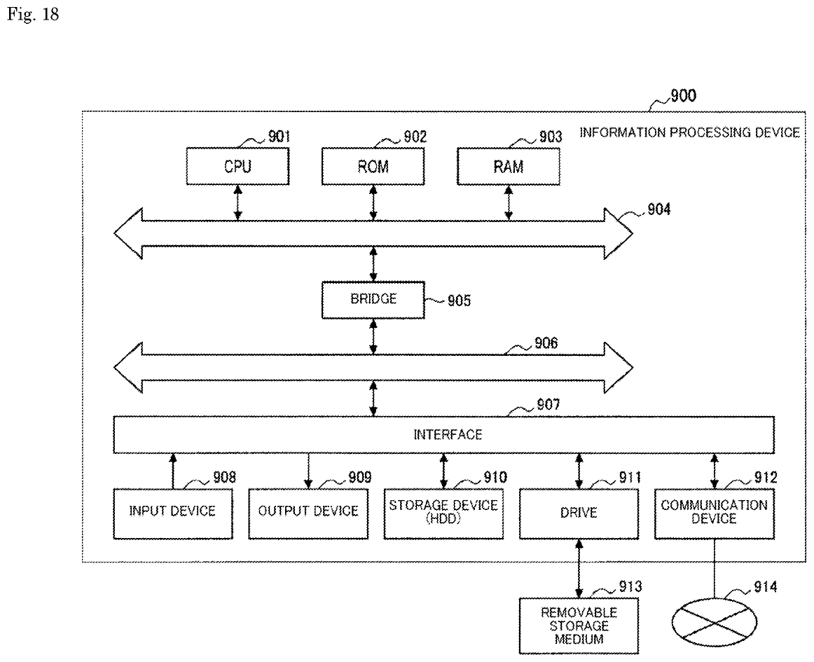

A specific example of a medical inquiry AI user interface has been described above. Next, an example of a hardware configuration of an information processing device 900, which realizes the matching server 100, the management server 200, the examination terminal 300, the user terminal 400, and the diagnosis server 600, will be described with reference to FIG. 18.

FIG. 18 shows the hardware configuration of the information processing device 900. The information processing device 900 includes a CPU (Central Processing Unit) 901, a ROM (Read Only Memory) 902, a RAM (Random Access Memory) 903, a host bus 904, a bridge 905, an external bus 906, an interface 907, an input device 908, an output device 909, a storage device (HDD) 910, a drive 911, and a communication device 912.

The CPU 901 functions as an arithmetic processing device and a control device, and performs overall control of operations in the information processing device 900 in accordance with various programs. The CPU 901 may also be a microprocessor. The ROM 902 stores programs, calculation parameters, and the like that are used by the CPU 901. The RAM 903 temporarily stores programs used in processing executed by the CPU 901, parameters that change appropriately in such execution, and the like. These members are connected to each other by the host bus 904, which is constituted by a CPU bus or the like. The functions of the processing unit 120 of the matching server 100, the processing unit 220 of the management server 200, the processing unit of the examination terminal 300 (not shown), the processing unit 420 of the user terminal 400, and the processing unit 620 of the diagnosis server 600 are realized through cooperation of the CPU 901, the ROM 902, and RAM 903.

The host bus 904 is connected to the external bus 906, which is a PCI (Peripheral Component Interconnect/Interface) bus, via the bridge 905. Note that it is not necessarily required for the host bus 904, the bridge 905, and the external bus 906 to be configured separately, and the functions thereof may be implemented by a single bus.

The input device 908 is configured by, for example, an input means for allowing the user to input information, such as a mouse, a keyboard, a touch panel, a button, a microphone, a switch, a lever, or a camera, and an input control circuit that generates input signals based on user input and outputs the input signals to the CPU 901. By operating the input device 908, the user of the information processing device 900 can input various types of information to the devices and give processing operation instructions. The functions of the input unit 440 of the user terminal 400 are realized by the input device 908.

The output device 909 includes a display device such as a CRT (Cathode Ray Tube) display device, a liquid crystal display (LCD) device, an OLED (Organic Light Emitting Diode) device, or a lamp. The output device 909 further includes an audio output device such as a speaker or headphones. The display device displays various types of information, such as video data, using text or images. The audio output device converts audio data or the like into audio and outputs the audio. The functions of the display unit 450 of the user terminal 400 are realized by the output device 909.

The storage device 910 is a device for data storage. The storage device 910 may include a storage medium, a recording device that records data on the storage medium, a reading device that reads data from the storage medium, a deleting device that deletes data recorded on the storage medium, and the like. The storage device 910 is constituted by an HDD (Hard Disk Drive), for example. The storage device 910 is drives the hard disk and stores programs executed by the CPU 901 and various types of data. The functions of the storage unit 130 of the matching server 100, the storage unit 230 of the management server 200, the storage unit of the examination terminal 300 (not shown), the storage unit 430 of the user terminal 400, and the storage unit 630 of the diagnosis server 600 are realized by the storage device 910.

The drive 911 is a storage medium reader/writer, and is built in or externally attached to the information processing device 900. The drive 911 reads information recorded on a mounted removable storage medium 913 such as a magnetic disk, an optical disc, a magneto-optical disk, or a semiconductor memory, and outputs the information to the RAM 903. The drive 911 can also write information to the removable storage medium 913.

The communication device 912 is, for example, a communication interface constituted by a communication device or the like for connecting to a communication network 914. The functions of the communication unit 110 of the matching server 100, the communication unit 210 of the management server 200, the communication unit of the examination terminal 300 (not shown), the communication unit 410 of the user terminal 400, and the communication unit 610 of the diagnosis server 600 are realized by the communication device 912.

### 5. Summary

As described above, the matching server 100 acquires an examination arrangement request and user information, and presents, to the user, examination resources available for performing examinations based on the acquired information. Also, the management server 200 receives an examination result from the examination terminal 300 after an examination has been performed using an examination resource, and manages the examination result in association with the user information. The management server 200 also outputs the examination result to a user-desired device based on user input. Through the above series of processing, the user can take various actions based on the examination result. For example, the user can make a self-diagnosis by outputting the examination result to their own device, or can use a diagnostic service by outputting the examination result to a device of a diagnostic service provider. This therefore reduces the number of hospital visits and the like by users who have mild symptoms, and reduces diagnostic personnel expenses for example, thus suppressing an increase in medical expenses.

Also, due to an examination arrangement request being generated by the medical inquiry AI, the user can easily request an arrangement for an examination resource. Moreover, a user symptom estimation can be made more easily and accurately by using the diagnosis AI 700.

Although preferred embodiments of the present disclosure have been described in detail above with reference to the accompanying drawings, the technical scope of the present disclosure is not limited to these examples. It is clear that various modifications and alterations can be arrived at by a person having ordinary knowledge in the technical field of the present disclosure within the scope of the technical idea described in the claims, and all of such modifications and alternations are also naturally understood to be included in the technical scope of the present disclosure.

Also, the effects described in the present specification are merely explanatory or illustrative, and are not intended to be limiting. In other words, a technique according to the present disclosure may exhibit other effects apparent to those skilled in the art from the description of the present specification, in addition to or in place of the above effects.

Configurations such as the following are also encompassed in the technical scope of the present disclosure.

(1)

A medical information processing system including:

an acquisition unit configured to acquire an examination arrangement request that includes examination type information that indicates a type of medical-related examination, and user information that is information regarding a user; a presentation unit configured to present, to the user, an examination resource capable of performing the examination, based on the examination arrangement request and the user information;
a management unit configured to manage a result of the examination performed on the user in association with the user information; and
an output unit configured to output the result based on input performed by the user.
(2)

The medical information processing system in section (1), wherein the examination resource does not perform a diagnosis, a treatment, a surgery, or an estimation of a symptom of the user based on the result.

(3)

The medical information processing system in section (2), wherein there are a plurality of the examination resources, including a medical examination institution, a medical examination worker, an examination device, and a test kit for use by the user.

(4)

The medical information processing system in section (2) or (3), wherein the output unit outputs the result to a device of an institution that performs a diagnosis, a treatment, a surgery, or an estimation of a symptom of the user based on the result.

(5)

The medical information processing system in section (2) or (3), further including: an estimation unit configured to make an estimation of a symptom of the user based on the result.

(6)

The medical information processing system in any one of sections (1) to (5), wherein

the user information includes at least one of position information regarding the user, historical information regarding an examination underwent by the user in the past, and setting information regarding an examination that the user is to undergo in the future.
(7)

The medical information processing system in any one of sections (1) to (6), wherein

the user information includes position information regarding the user, and the presentation unit extracts a plurality of examination resources capable of performing the examination based on the examination arrangement request, and extracts, from among the extracted examination resources, an examination resource that is to be presented based on the position information regarding the user and position information regarding the extracted examination resources.
(8)

The medical information processing system in section (7), wherein

the presentation unit extracts and present an examination resource that is located in a predetermined range of a position of the user based on the position information regarding the user and the position information regarding the examination resources that were extracted based on the examination arrangement request.
(9)

The medical information processing system in section (7), wherein

the presentation unit presents examination resources in order of highest proximity to the user based on the position information regarding the user and the position information regarding the examination resources that were extracted based on the examination arrangement request.
(10)

The medical information processing system in any one of sections (1) to (9), further including:

an arrangement unit configured to make an arrangement for the examination resource.
(11)

The medical information processing system in any one of sections (1) to (10), further including:

a generation unit configured to generate the examination arrangement request based on input performed by the user.
(12)

A medical information processing device including:

an acquisition unit configured to acquire an examination arrangement request that includes examination type information that indicates a type of medical-related examination, and user information that is information regarding a user; a presentation unit configured to present, to the user, an examination resource capable of performing the examination, based on the examination arrangement request and the user information;
a management unit configured to manage a result of the examination performed on the user in association with the user information; and an output unit configured to output the result based on input performed by the user.
(13)

A medical information processing method executed by a computer, the method including:

acquiring an examination arrangement request that includes examination type information that indicates a type of medical-related examination, and user information that is information regarding a user;
presenting, to the user, an examination resource capable of performing the examination, based on the examination arrangement request and the user information;
managing a result of the examination performed on the user in association with the user information; and
outputting the result based on input performed by the user.

### REFERENCE SIGNS LIST

100 Matching server
110 Communication unit
120 Processing unit
121 Authentication unit
122 Matching unit
123 Output unit
130 Storage unit
200 Management server
210 Communication unit
220 Processing unit
221 Authentication unit
222 Management unit
223 Arrangement unit
224 Output unit
230 Storage unit
300 Examination terminal
400 User terminal
410 Communication unit
420 Processing unit
421 Generation unit
430 Storage unit
440 Input unit
450 Display unit
500 Network
600 Diagnosis server
610 Communication unit
620 Processing unit
621 Management unit
622 Diagnosis AI cooperation unit
623 Output unit
630 Storage unit
700 Diagnosis AI

1. A medical information processing system comprising:
an acquisition unit configured to acquire an examination arrangement request that includes examination type information that indicates a type of medical-related examination, and user information that is information regarding a user;
a presentation unit configured to present, to the user, an examination resource capable of performing the examination, based on the examination arrangement request and the user information;
a management unit configured to manage a result of the examination performed on the user in association with the user information; and
an output unit configured to output the result based on input performed by the user.

2. The medical information processing system according to claim 1, wherein
the examination resource does not perform a diagnosis, a treatment, a surgery, or an estimation of a symptom of the user based on the result.

3. The medical information processing system according to claim 2, wherein
there are a plurality of the examination resources, including a medical examination institution, a medical examination worker, an examination device, and a test kit for use by the user.

4. The medical information processing system according to claim 2, wherein
the output unit outputs the result to a device of an institution that performs a diagnosis, a treatment, a surgery, or an estimation of a symptom of the user based on the result.

5. The medical information processing system according to claim 2, further comprising:
an estimation unit configured to make an estimation of a symptom of the user based on the result.

6. The medical information processing system according to claim 1, wherein
the user information includes at least one of position information regarding the user, historical information regarding an examination underwent by the user in the past, and setting information regarding an examination that the user is to undergo in the future.

7. The medical information processing system according to claim 1, wherein
the user information includes position information regarding the user, and the presentation unit extracts a plurality of examination resources capable of performing the examination based on the examination arrangement request, and extracts, from among the extracted examination resources, an examination resource that is to be presented based on the position information regarding the user and position information regarding the extracted examination resources.

8. The medical information processing system according to claim 7, wherein
the presentation unit extracts and present an examination resource that is located in a predetermined range of a position of the user based on the position information regarding the user and the position information regarding the examination resources that were extracted based on the examination arrangement request.

9. The medical information processing system according to claim 7, wherein
the presentation unit presents examination resources in order of highest proximity to the user based on the position information regarding the user and the position information regarding the examination resources that were extracted based on the examination arrangement request.

10. The medical information processing system according to claim 1, further comprising:
an arrangement unit configured to make an arrangement for the examination resource.

11. The medical information processing system according to claim 1, further comprising:
a generation unit configured to generate the examination arrangement request based on input performed by the user.

12. A medical information processing device comprising:
an acquisition unit configured to acquire an examination arrangement request that includes examination type information that indicates a type of medical-related examination, and user information that is information regarding a user;
a presentation unit configured to present, to the user, an examination resource capable of performing the examination, based on the examination arrangement request and the user information;
a management unit configured to manage a result of the examination performed on the user in association with the user information; and
an output unit configured to output the result based on input performed by the user.

13. A medical information processing method executed by a computer, the method comprising:
acquiring an examination arrangement request that includes examination type information that indicates a type of medical-related examination, and user information that is information regarding a user;
presenting, to the user, an examination resource capable of performing the examination, based on the examination arrangement request and the user information;
managing a result of the examination performed on the user in association with the user information; and
outputting the result based on input performed by the user.
